(19) United States
(12) Patent Application Publication
(10) Pub. No.: US 2024/0412873 A1
(43) Pub. Date: Dec. 12, 2024

(54) PRONING EFFICACY ASSESSMENT USING IMAGES

(71) Applicant: KONINKLIJKE PHILIPS N.V., Eindhoven (NL)

(72) Inventors: Cornelis Petrus Hendriks, Eindhoven (NL); Roberto Buizza, Malden, MA (US); Jaap Roger Haartssen, Best (NL); Thomas Koehler, Norderstedt (DE); Michael Polkey, London (GB); Joerg Sabczynski, Norderstedt (DE); Rafael Wiemker, Kisdorf (DE)

(21) Appl. No.: 18/588,281

(22) Filed: Feb. 27, 2024

Related U.S. Application Data
(60) Provisional application No. 63/471,276, filed on Jun. 6, 2023.

(51) Int. Cl. G16H 50/30; G16H 20/40; G16H 30/20

(52) U.S. Cl. CPC G16H 50/30; G16H 20/40; G16H 30/20

(57) ABSTRACT

A proning assessment method includes determining, from received respiratory and/or body position data, a flipping time (t_{flipping}) when a patient is placed in a partial or full prone position; determining a target imaging time (t_{img,target}) for acquiring imaging data to assess an impact of proning on mechanical ventilation therapy as the flipping time (t_{flipping}) plus a predetermined time interval (Δt); receiving at least one lung image of at least one lung of the patient, the at least one lung image being timestamped with an image acquisition time (t_{img,actual}); and at least one of: (i) displaying the at least one lung image; and/or (ii) analyzing the at least one lung image to generate a proning recommendation for the patient, and displaying, on a display device, a representation of the proning recommendation.

### CROSS-REFERENCE TO RELATED APPLICATIONS

This patent application claims the priority benefit under 35 U.S.C. § 119 (c) of U.S. Provisional Application No. 63/471,276, filed on Jun. 6, 2023, the contents of which are herein incorporated by reference.

The following relates generally to the respiratory therapy arts, mechanical ventilation arts, lung assessment arts, and related arts.

### BACKGROUND

Mechanically ventilated patients with certain respiratory conditions such as acute respiratory distress syndrome (ARDS) or COVID-19 may benefit from prone positioning (i.e., positioning the patient face down onto the patient's anterior chest and abdomen). With proning, oxygenation can improve, and mortality can be reduced due to an improved ventilation-perfusion match and potentially decreased lung injury. Physiological mechanisms that promote these beneficial effects are believed to include a redistribution of blood flow, a more homogeneous chest wall compliance, more optimally distributed gravitational forces on the lung parenchyma and surrounding organs such as the heart and the liver enabling recruitment of posterior lung regions, a more equal distribution of stress forces onto the lung by the diaphragm, and an enhanced inferior movement of the diaphragm.

However, proning an intubated patient undergoing mechanical ventilation therapy is not easy. The preparation may take half an hour and the actual flipping of the patient into the prone position requires a coordinated team effort at the bedside. Moreover, prone positioning may result in tubes and lines detaching (endotracheal tube, catheters), a decreased clearance of mucus, facial pressure ulcers, nerve injury and cardiopulmonary resuscitation.

Besides the burden of proning, not all patients respond well to proning, and currently clinicians have difficulty predicting which patients are most likely to improve with prone positioning. Even if the patient has previously had a positive (or negative) response to proning, this response may not be predictive of that patient's response to a future proning session. Studies have been undertaken to investigate the possibility of predicting proning success in terms of an improved oxygenation on the short term. For example, one study (Heldeweg et al., “Lung ultrasound to predict gas-exchange response to prone positioning in COVID-19 patients: A prospective study in pilot and confirmation cohorts”, Journal of Critical Care, Volume 73, 2023, 154173) has shown that a high lung ultrasound score prior to prone positioning correlates to a poor gas-exchange response. The ultrasound score is based on B-lines, consolidations, etc., with a higher index representing more aeration loss. Another study (Dam et al., “Predicting responders to prone positioning in mechanically ventilated patients with COVID-19 using machine learning”, Ann. Intensive Care 12, 99 (2022)) found that in mechanically ventilated Covid-19 patients, predicting the success of prone positioning using clinically relevant and readily available parameters from electronic health records is currently not feasible with the use of machine learning. Another study (Bell, J. et al., “Predicting Impact of Prone Position on Oxygenation in Mechanically Ventilated Patients with COVID-19”, Journal of Intensive Care Medicine 2022, Vol. 37(7) 883-889) focused on using clinical variables at the time of prone positioning, rather than on admission to the intensive care unit (ICU), initiation of mechanical ventilation, or during a patient's prior prone instance. Proning success was associated with a lower pre-prone PaO₂/FiO₂ ratio and receiving Erythropoietin (EPO) medication. In these studies, the number and duration of the prone sessions vary, and also the time between the response measurements and the patient position change.

A few methods are available to evaluate if proning is efficacious for a particular patient. These tests are applicable after the patient has been proned. In one example, a blood test is performed to measure whether the PaO₂/FiO₂ change is ≥20 mmHg. This parameter however is a poor surrogate for shunt and ventilation-perfusion mismatch. Another example is to observe if the pressure requirements go down, i.e., if a lower positive respiratory end pressure (PEEP) value is required to optimize oxygenation.

In another example, a chest x-ray is obtained. Changes in radiographic opacities correlate with outcomes on the short term (improved oxygenation) and the long term (mortality). Dynamic X-rays taken multiple times after proning can be performed to determine whether the lungs are opening, or the ventilation-perfusion matching improves.

The following discloses certain improvements to overcome these problems and others.

### SUMMARY

In one aspect, a respiration monitoring device includes at least one electronic processor programmed to perform a proning assessment method including receiving respiratory and/or body position data as a function of time of a patient as a function of time during inspiration and expiration while the patient undergoes mechanical ventilation therapy with a mechanical ventilator; determining, from the received respiratory and/or body position data, a flipping time (t_{flipping}) when the patient is placed in a partial or full prone position; determining a target imaging time (t_{img,target}) for acquiring imaging data to assess an impact of proning on the mechanical ventilation therapy as the flipping time (t_{flipping}) plus a predetermined time interval (Δt); receiving at least one lung image of at least one lung of the patient, the at least one lung image being timestamped with an image acquisition time (t_{img,actual}); and at least one of: (i) displaying the at least one lung image; and/or (ii) analyzing the at least one lung image to generate a proning recommendation for the patient, and displaying, on a display device, a representation of the proning recommendation.

In another aspect, a proning assessment method includes, with an electronic controller: receiving respiratory and/or body position data as a function of time of a patient as a function of time during inspiration and expiration while the patient undergoes mechanical ventilation therapy with a mechanical ventilator; determining, from the received respiratory and/or body position data, a flipping time (t_{flipping}) when the patient is placed in a partial or full prone position; determining a target imaging time (t_{img,target}) for acquiring imaging data to assess an impact of proning on the mechanical ventilation therapy as the flipping time (t_{flipping}) plus a predetermined time interval (Δt); receiving at least one lung image of at least one lung of the patient, the at least one lung image being timestamped with an image acquisition time (t_{img,actual}); and at least one of (i) displaying the at least one lung image; and/or (ii) analyzing the at least one lung image to generate a proning recommendation for the patient, and displaying, on a display device, a representation of the proning recommendation.

One advantage resides in determining a benefit of proning a patient undergoing mechanical ventilation therapy,

Another advantage resides in predicting a patient response to proning while undergoing mechanical ventilation therapy.

Another advantage resides in providing accurate data to determine an effect of proning a patient undergoing mechanical ventilation therapy.

Another advantage resides in providing a patient-specific model to determine a benefit of proning a patient undergoing mechanical ventilation therapy.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows an illustrative respiration monitoring device in accordance with the present disclosure.

FIGS. 2 and 3 show example flow charts of operations suitably performed by the system of FIG. 1.

### DETAILED DESCRIPTION

As used herein, the singular form of “a,” “an,” and “the” include plural references unless the context clearly dictates otherwise. As used herein, statements that two or more parts or components are “coupled,” “connected,” or “engaged” shall mean that the parts are joined, operate, or co-act together either directly or indirectly, i.e., through one or more intermediate parts or components, so long as a link occurs. Directional phrases used herein, such as, for example and without limitation, top, bottom, left, right, upper, lower, front, back, and derivatives thereof, relate to the orientation of the elements shown in the drawings and are not limiting upon the scope of the claimed invention unless expressly recited therein. The word “comprising” or “including” does not exclude the presence of elements or steps other than those described herein and/or listed in a claim. In a device comprised of several means, several of these means may be embodied by one and the same item of hardware.

The following discloses using diaphragmatic ultrasound and/or ventilator waveforms to evaluate and predict a patient response to prone positioning. Diaphragmatic ultrasound is a non-invasive imaging modality that is readily available in the ICU which is used for visualization of the diaphragm. Diaphragmatic ultrasound allows a clinician to determine the thickness and thickening fraction of the diaphragm from which respiratory activity and hence the onset of inspiration can be derived to evaluate and predict a patient response to prone positioning.

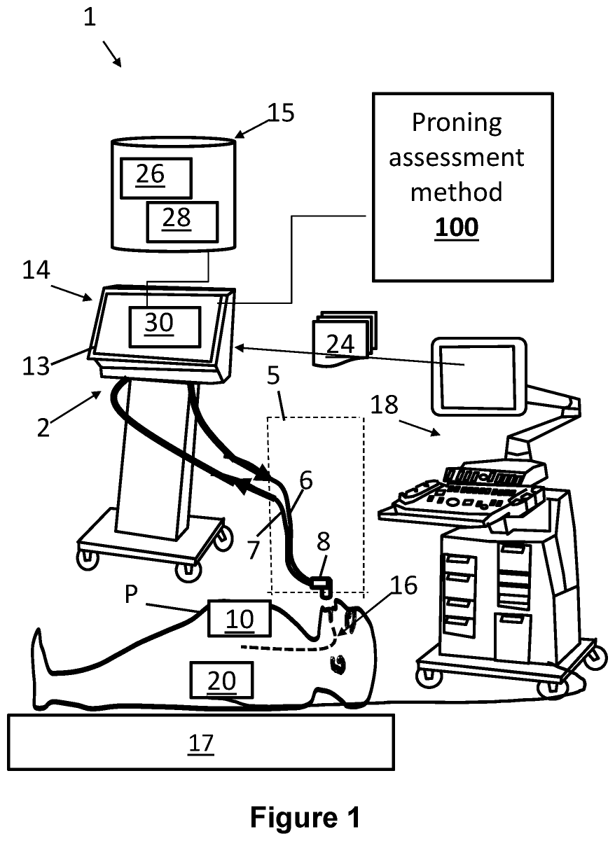

With reference to FIG. 1, a respiration monitoring device 1 is shown. A mechanical ventilator 2 is configured to provide ventilation therapy to an associated patient P is shown. As shown in FIG. 1, the mechanical ventilator 2 is connected with a patient breathing circuit 5 to deliver mechanical ventilation to the patient P. The patient breathing circuit 5 includes typical components for a mechanical ventilator, such as an inlet line 6 (also called inhalation limb 6), an optional outlet line 7 (also called exhalation limb 7; this may be omitted if the ventilator employs a single-limb patient circuit), a connector or port 8 for connecting with an endotracheal tube (ETT) 16, or a mask, and one or more breathing sensors (not shown), such as a gas flow meter, a pressure sensor, end-tidal carbon dioxide (etCO₂) sensor, and/or so forth. The mechanical ventilator 2 is designed to deliver air, an air-oxygen mixture, or other breathable gas (supply not shown) to the inhalation limb 6 of the patient breathing circuit 5 at a programmed pressure and/or flow rate to ventilate the patient via an ETT, and optionally also handling exhaled air received at the mechanical ventilator 2 via the exhalation limb 7 of the patient breathing circuit 5. The mechanical ventilator 2 also includes at least one electronic processor or controller 13 (e.g., an electronic processor or a microprocessor), a display device 14, and a non-transitory computer readable medium 15 storing instructions executable by the electronic controller 13.

FIG. 1 diagrammatically illustrates the patient P intubated with an ETT 16 (most of which is inside the patient P and hence is shown in phantom). The connector or port 8 connects with the ETT 16 to operatively connect the mechanical ventilator 2 to deliver breathable air to the patient P via the ETT 16. The mechanical ventilation provided by the mechanical ventilator 2 via the ETT 16 may be therapeutic for a wide range of conditions, such as various types of pulmonary conditions like emphysema or pneumonia, viral or bacterial infections impacting respiration such as a COVID-19 infection or severe influenza, cardiovascular conditions in which the patient P receives breathable gas enriched with oxygen, or so forth.

FIG. 1 illustrates the patient P positioned in the supine position on a diagrammatically indicated patient bed or other support 17. In the supine position, the patient P is lying face-up (unless the head is tilted), that is, with the patient lying on his or her back. The patient P is typically intubated with the patient in the supine position, as shown in FIG. 1, as the supine position places the face of the patient facing generally upward, away from the bed 17, thus providing the clinician with access to the patient's mouth to perform the intubation. After intubation, the patient commonly remains in the supine position while receiving the mechanical ventilation therapy. The supine position beneficially keeps the connector or port 8 and the portion of the ETT 16 extending outside of the patient's mouth away from the bed 17 and facilitates periodical clinician check of the condition of the ETT 16 and connector or port 8.

As previously discussed, however, sometimes the patient may benefit from being flipped over into the prone position (i.e., lying face-down, with the patient lying on his or her chest). The act of repositioning (i.e., flipping) the patient into the prone position is referred to as proning. Proning can improve oxygenation and reduce mortality for some patients. Without being limited to any particular theory of operation, it is believed these benefits arise due to benefits of the prone position such as redistribution of blood flow, more homogeneous chest wall compliance, more optimally distributed gravitational forces on the lung parenchyma and surrounding organs such as the heart and the liver enabling recruitment of posterior lung regions, a more equal distribution of stress forces onto the lung by the diaphragm, and an enhanced inferior movement of the diaphragm.

However, not all mechanically ventilated patients benefit from being placed into the prone position. Proning may not be beneficial for some patients and may even reduce effectiveness of the mechanical therapy. Moreover, the practical benefits of the supine position (e.g., easy clinician access to the ETT 16 and connector or port 8, avoiding interference between these components and the patient bed 17, and so forth) are lost when the patient is proned. Hence, the clinician would benefit from a quantitative and standardized approach for assessing the likely efficacy (or lack thereof) of proning prior to or shortly after the patient is proned. In approaches disclosed herein, medical imaging is used to provide such assessment.

To this end, FIG. 1 also shows a medical imaging device 18 (also referred to as an image acquisition device, imaging device, and so forth). the illustrative medical imaging device 18 comprises an ultrasound (US) medical imaging device 18. However, more generally the medical imaging device could be an ultrasound imaging device (as illustrated), an X-ray imaging device, a computed tomography (CT) imaging device, a magnetic resonance imaging (MRI) device, or so forth. In some embodiments, the imaging device is a portable bedside imaging device (such as the illustrative ultrasound imaging device 18, or a portable X-ray imaging device) which beneficially enables the patient P to be images lying in the bed 17, in either the illustrated supine position (for images acquired before flipping) or in the prone position (for images acquired after flipping).

The illustrative ultrasound medical imaging device 18 includes an ultrasound probe or patch 20 configured to acquire US imaging data (i.e., US images) 24 of one or both lungs of the patient P. Typically the ultrasound probe 20 would be positioned near each lung (of the left and right lungs) in turn to image the two lungs. On the other hand, if the imaging device 18 is an X-ray imaging device, then an image of both left and right lungs may be captured in a single X-ray image. The electronic processor 13 controls the ultrasound (or other-modality) imaging device 18 to receive the imaging data 24 of the diaphragm of the patient P from the US patch 20.

The non-transitory computer readable medium 15 stores instructions executable by the electronic controller 13 to perform a proning assessment method or process 100.

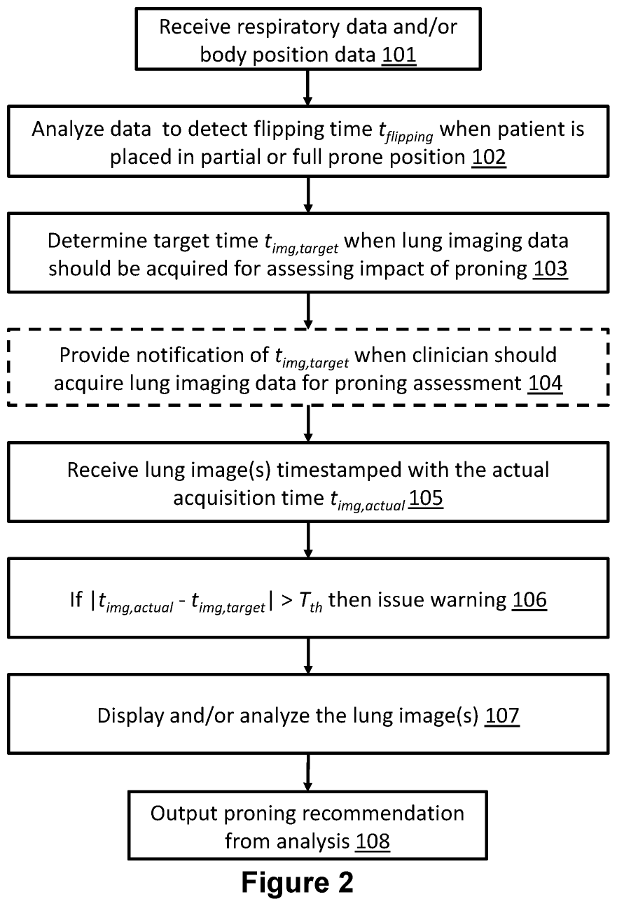

With reference to FIG. 2, and with continuing reference to FIG. 1, an illustrative embodiment of the proning assessment method 100 is diagrammatically shown as a flowchart. At an operation 101, respiratory and/or body position data of the patient P is received at the electronic controller 13 as a function of time during inspiration and expiration while the patient P undergoes mechanical ventilation therapy with the mechanical ventilator 2. The respiratory data of the patient P can include one or more of an airway pressure in an airway of the patient P or an airway flow in an airway of the patient P measured during ventilation therapy by the mechanical ventilator 2. Airway pressure and airway flow are commonly monitored during mechanical ventilation, so this data is typically available for any mechanically ventilated patient.

As recognized herein, accurate proning assessment by medical imaging can be sensitive to the timing of the acquisition of the lung image(s) used in the assessment. Specifically, it is beneficial to employ a standard time between when the patient P is placed into the prone position (i.e. when the patient is flipped, denoted herein as flipping time t_{flipping}) and can occur throughout a day), and the time when the lung image(s) for the proning assessment are acquired. However, standardization of timing can be difficult to achieve. In some mechanical ventilation therapy protocols, the patient P is imaged bedside at relatively regular intervals, e.g., once a day. While this might be expected to provide a standard time interval, in practice the mobile imaging device 18 used for the imaging may be employed in many different patient rooms each day, and so the time of imaging each day can vary by a few hours or more. If an imaging modality such as CT or MRI is used, the patient P must be transported to the imaging device 18 (e.g., located in a dedicated radiology department) which makes standardization of timing of the acquisition even more challenging. As discussed below, the proning assessment method 100 includes approaches for addressing this.

At an operation 102, a flipping time (t_{flipping}) when the patient is placed in a partial or full prone position is determined from the received respiratory and/or body position data. In one example, the received data in the operation 101 is respiratory data, and the flipping time t_{flipping} is determined based on a detected time interval in the respiratory data as a function of time during which valid respiratory data is not received.

In another example, the received data in the operation 101 is body position data, and the flipping time t_{flipping} is determined as a time when the body position data indicates the patient is placed into a partial or full prone position. To acquire the body position data, a sensor 10 (e.g., a three-axis accelerometer or gyroscope in a patch or a belt) can be attached to the patient P to measure body position data of the patient P. The body position, and changes in body position including the time stamps, can be directly determined from an accelerometer signal. The body position can be determined in an accurate manner including positions between supine and proning, e.g., lateral, inclined, etc.

During flipping, waveforms from the mechanical ventilator 2 (or a patient monitor) show disruptions of different kinds, such as, for example, a short drop in pressure and flow due to the ventilation pause, a step or a change in vital signs, or a combination thereof. Univariate or multivariate signal processing methods can be used to detect these features such as, for example, threshold detection, autocorrelation, Fourier transformation, filtering, machine learning, and so forth. When a feature has been identified in the waveform, the time stamp of this corresponding data point is extracted, t_{flipping}. This time stamp is used to determine the time between the start of proning and the image acquisition, Δt=t_{image acquisition}−t_{flipping}.

The waveforms can also be disrupted due to endotracheal tube cleaning, replacement, or dislodgement or due to other events such as a heart attack. This can be distinguished from patient flipping by comparing the characteristic features of flipping and other events, and/or by comparing the signals before and after the event.

In the case of multiple proning episodes, more than one patient flipping feature can be identified in the waveforms. This information can be used to determine the history of proning episodes, for example the number, the duration, and the intervals.

At an operation 103, a target imaging time t_{img,target} for acquiring imaging data 24 is determined to assess an impact of proning on the mechanical ventilation therapy as the flipping time t_{flipping} plus a predetermined time interval Δtp, i.e., t_{img,target}=t_{flipping}+Δtp. The predetermined time interval Δt is typically a standard time interval after the flipping for images to be acquired for evaluating the efficacy of the proning. The time interval Δt may, for example, be the time interval that pulmonologists have empirically determined is long enough for the proning benefits to accrue to a sufficient extent to be detectable in the imaging. In some embodiments to be described, the proning assessment may use an artificial intelligence (AI) model—in such cases, the time interval Δt is typically the time interval after flipping at which training images used in training the AI model were acquired. Ideally, the lung image(s) for proning assessment should be acquired at the target imaging time t_{img,target}=t_{flipping}+Δtp.

At an optional operation 104 (depicted in FIG. 2 with dashed lines), a notification 30 can be output on the display device 14 for acquiring imaging data 24 to assess the impact of proning on the mechanical ventilation therapy. This can be performed at or prior to the target imaging time t_{img,target}, or both prior to t_{img,target} and at t_{img,target}. For example, one notification may be issued a set time before t_{img,target} to provide advance notification to enable clinicians to prepare (for example, by locating the portable imaging device 18 and moving it into the hospital room of the patient P), followed by a second notification issued at t_{img,target} to inform clinicians of exactly when the lung image(s) should be acquired.

At an operation 105, at least one lung image 24 of at least one lung of the patient P is received by the electronic controller 13 and is timestamped with an image acquisition time (t_{img,actual}). Again, the electronic controller 13 can control the ultrasound patch 20 to acquire the ultrasound imaging data 24 and receive the ultrasound imaging data 24 of the diaphragm of the patient P from the ultrasound patch 20.

As previously noted, ideally the actual imaging time should be the target imaging time, that is, ideally t_{img,actual}=t_{img,target}. In practice, however, this will usually not be the case, as the practicalities of moving the imaging device 18 to the patient P (or vice versa), setting up for the imaging, and acquiring the lung image(s) for the proning evaluation take time and can be delayed by various other hospital operations and clinician tasks. However, if the actual imaging time is close enough to the target imaging time, then the lung image(s) should be usable. For example, if the difference is one minute this is unlikely to matter; but if the difference is ten hours that could make the proning assessment unreliable. Hence, at an operation 106, a time difference between the target imaging time f_{img,target} and the image acquisition time t_{img,actual} is computed. If the computed time difference is greater than a threshold Tₜₕ, then a warning 30 is output on the display device 14. That is, if |t_{img,actual}−t_{img,target}|>Tₜₕ then the warning 30 is output. In this expression, | . . . | is absolute value. The threshold Tₜₕ is chosen to be small enough to ensure that the proning assessment using the acquired lung image(s) is likely to be accurate, but large enough to accommodate practical limitations in the timing precision of the clinician workflow.

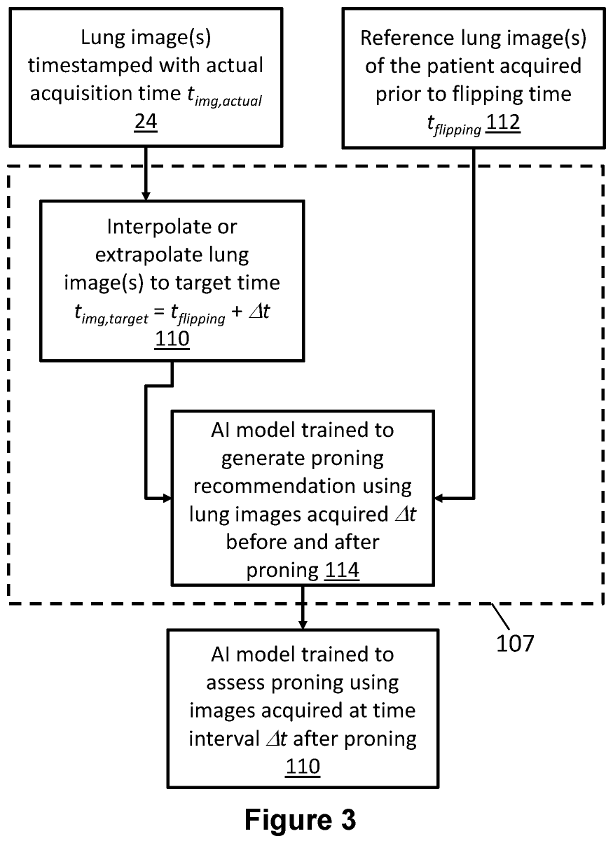

At an operation 107, the at least one lung image 24 is displayed, and/or analyzed to generate a proning recommendation for the patient P. In one embodiment, the operation 107 is a display operation in which the at least one lung image 24 received in the operation 105 is displayed, optionally along with displaying at least one reference lung image acquired prior to the proning of the patient. (This reference lung image could correspond to a reference lung image 112 described later herein with reference to FIG. 3). In this way, a clinician can visually review the received at least one lung image 24 (and optionally compare it with the reference lung image) to manually perform a proning assessment.

In another embodiment, the operation 107 performs an automated analysis of the at least one lung image 24. For example, the analysis could include applying an artificial intelligence (AI) model, as will be described in further detail in the embodiment of FIG. 3. In this case, the operation 107 outputs a proning recommendation generated by the analysis, and in an operation 108, the proning recommendation is output, e.g. on a display. The proning recommendation could, for example be “Proning should be continued” or “Proning should be discontinued.” Optionally, the proning recommendation could include a basis for the recommendation, such as “The image indicates <specified improvement>” to support a recommendation to continue proning. In addition to outputting the proning recommendation, the at least one lung image 24 could also be displayed so the clinician can also manually review it.

In the embodiment of FIG. 2, the operation 106 assesses whether the actual imaging time t_{img,actual} is sufficiently close to the target imaging time t_{img,target} for the proning assessment using the at least one lung image to be reliable. In other embodiments, the display or analysis operation 107 interpolation or extrapolation is used to approximate an image at the target imaging time t_{img,target} from one or two images acquired near to, but not at, the target imaging time t_{img,target}.

Referring now to FIG. 3, another embodiment of the operation 107 is diagrammatically shown as a flowchart. FIG. 3 depicts an embodiment where the operation 107 includes generating the proning recommendation by inputting the at least one lung image 24 to a trained artificial intelligence (AI) model 28. The trained AI model 28 is trained on training data comprising lung images of historical patients before proning or placed in partial or full prone position acquired at the predetermined time interval (41) after the respective historical patients were placed into the partial or full prone position.

In the approach of FIG. 3, the at least one lung image 24 acquired at the operation 105 is optionally interpolated or extrapolated to the target imaging time t_{img,target}, as shown at the operation 110 from a computed time difference between the target imaging time (t_{img,target}) and the image acquisition time (t_{img,actual}). If images 24 are available only at points in time which are different from the time of flipping, the interpolation of images can be used to generate an image which corresponds to the patient status at a fixed number of hours after the start or the end of a proning episode (that is, at the target imaging time t_{img,target}).

For example, the at least one lung image 24 includes a preceding lung image timestamped with an image acquisition time preceding the target imaging time (t_{img,target}) and a succeeding lung image timestamped with an image acquisition time that is after the target imaging time (t_{img,target}). In one embodiment, the preceding and succeeding lung images are interpolated to produce an interpolated lung image corresponding to the target imaging time (t_{img,target}), and the interpolated lung image is analyzed to generate the proning recommendation for the patient P. The analysis can include comparing the interpolated image with the reference lung image (which can also be the preceding image). These images can be displayed and be analyzed by the clinician.

Optionally, at least one reference lung image 112 of the at least one lung of the patient P that is acquired prior to the flipping time (t_{flipping}) is received. At an operation 114, the proning recommendation is generated by inputting the at least one reference lung image 112, to the trained AI model 28. In some embodiments, the proning recommendation is generated based on a comparison of the at least one lung image (either interpolated as in FIG. 3, or as-acquired at the actual imaging time t_{img,actual} if it is close enough to the target imaging time t_{img,target} as in the example of FIG. 2) and the at least one reference lung image 112. The output of applying the trained AI model 28 in operation 114 is the proning recommendation, which is output per operation 108 of FIG. 2.

At an operation 110, the trained AI model 28 can be updated after the patient P is moved during the mechanical ventilation therapy. The AI model 28 can be used to assess proning using images acquired at time interval Δt after proning. In some examples, the AI model 28 can be updated with movement data obtained by the sensor 10.

Referring back to FIG. 2, at an operation 110, a representation 30 of the proning recommendation is displayed on the display device. In some examples, the information obtained by the waveform analysis (Δt, proning history) is displayed in the form of labels in the GUI 28 which displays the medical image. In other examples, if the patient P was placed in a partially prone position, then the recommendation 30 can be a recommendation to place the patient P in a fully prone position.

In some embodiments, the mechanical ventilator 2 can be controlled to adjust one or more parameters of the mechanical ventilation therapy delivered to the patient based on the proning recommendation. For example, an adjustment of at least one mechanical ventilation setting of the mechanical ventilator 2 can be determined based on the proning recommendation. The determined adjustment can be applied to the mechanical ventilator 2 to adjust the mechanical ventilation therapy to the patient P.

In some embodiments, the operation 107 performs analysis to determine a proning recommendation using a patient specific biophysical model 26 (stored in the non-transitory computer readable medium 15) which can be used to simulate the ventilation and perfusion (VQ) distribution in the lungs of the patient P to optimize a ventilation treatment (see, e.g., Burrowes, K. S. and Tawhai, M. H., 2006, “Computational predictions of pulmonary blood flow gradients: Gravity versus structure”, Respiratory Physiology & Neurobiology, 154 (2006) 515-523; Burrowes, K. S., et al., 2017, “Image-based computational fluid dynamics in the lung: virtual reality or new clinical practice?”, WIREs Syst Biol Med 2017, 9: e1392.). To do so, at least two medical images (e.g., dynamic X-ray, EIT, VQ scan, MRI, SPECT, PET) providing the ventilation and perfusion distribution acquired at two arbitrary points in time before and after flipping, can be used to validate the patient specific biophysical model 26. The validated patient model 26 can then be used to support clinical decisions on future proning based on a lung image acquired before flipping. The patient model 26 predicts the effect of proning on the ventilation and perfusion distribution. The model output (e.g., the redistribution of blood flow, VQ matching, oxygenation) can be used to decide if, when and how long the patient should be proned.

As an example, when the model 26 predicts a blood flow redistribution to diseased lung areas, proning might be less effective. On the other hand, when the blood flows to ventilated areas, proning might be effective. Depending on the simulation outcome the recommendation is “prone” or “not prone.”

In the previous examples, the proning operation is assumed to entail flipping the patient 180 degrees, from the supine (face-up) position to the prone (face-down) position. In some variant embodiments, before deciding to prone the patient P, a patient manipulation might be used to validate the biophysical model 26. For example, a clinician can move the patient P to tilt the head or the upper body, or only flip the patient 90 degrees while taking an X-ray image. This patient manipulation can be viewed as partial proning. The validated model 26 can then be used to predict the effect of a full proning. If the model prediction points out that proning is not so effective for the patient P, then full proning can be omitted which saves a lot of effort for the staff and risks for the patient.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A respiration monitoring device, comprising:
at least one electronic processor programmed to perform a proning assessment method including:
receiving respiratory and/or body position data as a function of time of a patient as a function of time during inspiration and expiration while the patient undergoes mechanical ventilation therapy with a mechanical ventilator;
determining, from the received respiratory and/or body position data, a flipping time (t_{flipping}) when the patient is placed in a partial or full prone position;
determining a target imaging time (t_{img,target}) for acquiring imaging data to assess an impact of proning on the mechanical ventilation therapy as the flipping time (t_{flipping}) plus a predetermined time interval (Δt);
receiving at least one lung image of at least one lung of the patient, the at least one lung image being timestamped with an image acquisition time (t_{img,actual}); and
at least one of:
(i) displaying the at least one lung image; and/or
(ii) analyzing the at least one lung image to generate a proning recommendation for the patient, and displaying, on a display device, a representation of the proning recommendation.

2. The device of claim 1, wherein the receiving comprises:
receiving respiratory data as a function of time of the patient and the flipping time (t_{flipping}) is determined based on a detected time interval in the respiratory data as a function of time during which valid respiratory data is not received.

3. The device of claim 1, wherein the receiving comprises:
receiving body position data as a function of time of the patient and the flipping time (t_{flipping}) is determined as a time when the body position data indicates the patient is placed into a partial or full prone position.

4. The device of claim 1, wherein the proning assessment method further includes:
at or prior to the target imaging time (t_{img,target}), outputting a notification of the target imaging time (t_{img,target}) for acquiring imaging data to assess the impact of proning on the mechanical ventilation therapy.

5. The device of claim 1, wherein the method includes analyzing the at least one lung image to generate a proning recommendation for the patient, and the analyzing includes:
computing a time difference between the target imaging time (t_{img,target}) and the image acquisition time (t_{img,actual}); and
interpolating or extrapolating the at least one lung image to the target imaging time (t_{img,target}) based on the time difference.

6. The device of claim 5, wherein:
the at least one lung image includes a preceding lung image timestamped with an image acquisition time preceding the target imaging time (t_{img,target}) and a succeeding lung image timestamped with an image acquisition time that is after the target imaging time (t_{img,target});
the preceding and succeeding lung images are interpolated to produce an interpolated lung image corresponding to the target imaging time (t_{img,target}); and
the interpolated lung image is analyzed to generate the proning recommendation for the patient.

7. The device of claim 5, wherein:
the at least one lung image is extrapolated to the target imaging time (t_{img,target}) to produce an extrapolated lung image corresponding to the target imaging time (t_{img,target}); and
the extrapolated lung image is analyzed to generate the proning recommendation for the patient.

8. The device of claim 1, further comprising:
computing a time difference between the target imaging time (t_{img,target}) and the image acquisition time (t_{img,actual}); and
outputting a warning that the proning recommendation may be unreliable if the time difference is greater than a threshold.

9. The device of claim 1, wherein the method includes analyzing the at least one lung image to generate a proning recommendation for the patient, and the analyzing comprises:
generating the proning recommendation by inputting the at least one lung image to a trained artificial intelligence (AI) model;
wherein the trained AI model is trained on training data comprising lung images of historical patients placed in partial or full prone position.

10. The device of claim 9, wherein the proning assessment method further includes:
receiving at least one reference lung image of the at least one lung of the patient, the at least one reference lung image being acquired prior to the flipping time (t_{flipping}); and
the proning recommendation is generated by inputting both the at least one lung image and the at least one reference lung image to the trained AI model.

11. The device of claim 9, wherein the proning assessment method further includes:
updating the trained AI model after the patient is moved during the mechanical ventilation therapy.

12. The device of claim 1, wherein the method includes analyzing the at least one lung image to generate a proning recommendation for the patient, and the analyzing includes:
receiving at least one reference lung image of the at least one lung of the patient, the at least one reference lung image being acquired prior to the flipping time (t_{flipping});
wherein the proning recommendation is generated based on a comparison of the at least one lung image and the at least one reference lung image.

13. The device of claim 12, wherein the proning assessment method includes:
obtaining, with a sensor attached to the patient, movement data of the patient; and
updating the trained model with the obtained movement data.

14. The device of claim 1, wherein the proning assessment method further includes displaying the at least one lung image.

15. A proning assessment method comprising, with an electronic controller:
receiving respiratory and/or body position data as a function of time of a patient as a function of time during inspiration and expiration while the patient undergoes mechanical ventilation therapy with a mechanical ventilator;
determining, from the received respiratory and/or body position data, a flipping time (t_{flipping}) when the patient is placed in a partial or full prone position;
determining a target imaging time (t_{img,target}) for acquiring imaging data to assess an impact of proning on the mechanical ventilation therapy as the flipping time (t_{flipping}) plus a predetermined time interval (Δt);
receiving at least one lung image of at least one lung of the patient, the at least one lung image being timestamped with an image acquisition time (t_{img,actual}); and
at least one of:
(i) displaying the at least one lung image; and/or
(ii) analyzing the at least one lung image to generate a proning recommendation for the patient, and displaying, on a display device, a representation of the proning recommendation.
